(19) United States
(12) Patent Application Publication
(10) Pub. No.: US 2023/0190198 A1
(43) Pub. Date: Jun. 22, 2023

(54) METHODS FOR FORMING A SPLINE USING A FLEXIBLE CIRCUIT ASSEMBLY AND ELECTRODE ASSEMBLIES INCLUDING SAME

(71) Applicant: St. Jude Medical, Cardiology Division, Inc., St. Paul, MN (US)

(72) Inventors: Brian PEDERSON, Selma, TX (US); Greg Olson, Elk River, MN (US)

(21) Appl. No.: 17/995,260

(22) PCT Filed: Mar. 29, 2021
(86) PCT No.: PCT/US2021/024643

§ 371 Date: Sep. 30, 2022

Related U.S. Application Data
(60) Provisional application No. 63/021,737, filed on May 8, 2020.

(51) Int. Cl. A61B 5/00; A61B 18/14

(52) U.S. Cl. CPC A61B 5/6858; A61B 18/1492; A61B 2017/00526

(57) ABSTRACT

A method of forming a spline for an electrode assembly includes providing a structural member including a first surface and a second surface. The method also includes providing a flexible circuit assembly including a plurality of electrodes and at least one flexible circuit substrate having a contact surface and an outer surface opposite the contact surface. The plurality of electrodes are disposed on the outer surface of the at least one flexible circuit substrate. The method includes positioning the flexible circuit assembly relative to the structural member such that a first set of electrodes is aligned with the first surface and a second set of electrodes is aligned with the second surface. The method also includes coupling the at least one flexible circuit substrate to at least one of the structural member and the at least one flexible circuit substrate.

### CROSS-REFERENCE TO RELATED APPLICATIONS

This application claims priority to U.S. Provisional Patent Application Ser. No. 63/021,737, filed May 8, 2020, the disclosure of which is hereby incorporated by reference in its entirety.

### BACKGROUND OF THE DISCLOSURE

### a. Field of the Disclosure

The present disclosure relates generally to medical devices that are used in the human body. In particular, the present disclosure relates to methods of forming splines for electrode assemblies using flexible circuit assemblies.

### b. Background

Electrophysiology catheters are used in a variety of diagnostic, therapeutic, and/or mapping and ablative procedures to diagnose and/or correct conditions such as atrial arrhythmias, including for example, ectopic atrial tachycardia, atrial fibrillation, and atrial flutter.

Typically, to perform such diagnostic, therapeutic, and/or mapping and ablative procedures, a catheter is deployed and manipulated through a patient's vasculature to an intended site, for example, a site within a patient's heart. The catheter typically carries one or more electrodes that can be used for cardiac mapping or diagnosis, ablation, and/or other therapy delivery modes, or both, for example. Ablation therapy may be used to treat various conditions afflicting the human anatomy, including atrial or cardiac arrhythmias. When tissue is ablated, or at least subjected to ablative energy generated by an ablation generator and delivered by an ablation catheter, lesions form in the tissue. Electrodes mounted on or in ablation catheters are used to create tissue necrosis in cardiac tissue to correct conditions such as atrial arrhythmia (including, but not limited to, ectopic atrial tachycardia, atrial fibrillation, and atrial flutter). Arrhythmias can create a variety of dangerous conditions including loss of synchronous atrioventricular contractions and stasis of blood flow. It is believed that the primary cause of atrial arrhythmia is stray electrical signals within the left or right atrium of the heart. The ablation catheter imparts ablative energy (e.g., radiofrequency energy, cryoablation, lasers, chemicals, high-intensity focused ultrasound, etc.) to cardiac tissue to create a lesion in the cardiac tissue. This lesion disrupts undesirable electrical pathways and thereby limits or prevents stray electrical signals that lead to arrhythmias.

Electroporation is a non-thermal ablation technique that involves applying strong electric-fields that induce pore formation in the cellular membrane. The electric field may be induced by applying a relatively short duration pulse which may last, for example, from a nanosecond to several milliseconds. Such a pulse may be repeated to form a pulse train. When such an electric field is applied to tissue in an in vivo setting, the cells in the tissue are subjected to a trans-membrane potential, which opens the pores on the cell wall. Electroporation may be reversible (i.e., the temporally-opened pores will reseal) or irreversible (i.e., the pores will remain open), causing cellular destruction. For example, in the field of gene therapy, reversible electroporation is used to transfect high molecular weight therapeutic vectors into the cells. In other therapeutic applications, a suitably configured pulse train alone may be used to cause cell destruction, for instance by causing irreversible electroporation.

Catheters, such as basket catheters and planar catheters, have electrodes distributed along a set number of splines. In particular, electrodes are typically disposed on one side of each spline. Thus, the electrode density of at least some known catheters is limited by the number of splines and the number of electrodes disposed on each spline. Electrode assemblies can be limited to a set number of splines due to inherent difficulties in increasing the number of splines. For example, with respect to basket catheters, as a number of splines increases, the diameter of the electrode basket increases, which is not desirable as larger electrodes baskets may be more difficult to deploy in smaller target locations. Alternatively, narrower splines can be used to maintain the diameter of the electrode basket, but narrower splines limit electrode size.

Additionally, at least some known catheters result in a positioning force only being applied on one side of the catheter when deployed. For example, spiral catheters are necessarily attached only at one point (e.g., at a proximal end thereof), which results in the positioning force being applied on only one side of the spiral. Thus, force cannot be applied on the opposite side (e.g., 180° around the spiral).

### SUMMARY OF THE DISCLOSURE

The present disclosure is directed to a method of forming a spline for an electrode assembly for a catheter system. The method includes providing a structural member including a first surface and a second surface. The method also includes providing a flexible circuit assembly including a plurality of electrodes and at least one flexible circuit substrate having a contact surface and an outer surface opposite the contact surface. The plurality of electrodes are disposed on the outer surface of the at least one flexible circuit substrate. The method includes positioning the flexible circuit assembly relative to the structural member such that a first set of electrodes is aligned with the first surface and a second set of electrodes is aligned with the second surface. The method also includes coupling the at least one flexible circuit substrate to at least one of the structural member and the at least one flexible circuit substrate.

The present disclosure is further directed to an electrode assembly for a catheter system. The electrode assembly has a longitudinal axis, a proximal end, and a distal end. The electrode assembly includes at least one spline extending from the proximal end to the distal end of the electrode assembly. The at least one spline includes a structural member extending from the proximal end to the distal end of the electrode assembly. The structural member includes a first surface and a second surface. The at least one spline also includes a flexible circuit assembly including a plurality of electrodes and at least one flexible circuit substrate having a contact surface and an outer surface opposite the contact surface. The plurality of electrodes are disposed on the outer surface of the at least one flexible circuit substrate. The flexible circuit assembly is positioned relative to the structural member such that a first set of electrodes of the plurality of electrodes is aligned with the first surface of the structural member and a second set of electrodes of the plurality of electrodes is aligned with the second surface of the structural member. The at least one flexible circuit substrate is coupled to at least one of the structural member and the at least one flexible circuit substrate.

The present disclosure is further directed to a catheter system including a flexible catheter shaft, a handle coupled to a proximal end of the catheter shaft, and an electrode assembly. The electrode assembly is coupled to a distal end of the flexible catheter shaft and has a longitudinal axis, a proximal end, and a distal end. The electrode assembly includes at least one spline extending from the proximal end to the distal end of the electrode assembly. The at least one spline includes a structural member extending from the proximal end to the distal end of the electrode assembly. The structural member includes a first surface and a second surface. The at least one spline also includes a flexible circuit assembly including a plurality of electrodes and at least one flexible circuit substrate having a contact surface and an outer surface opposite the contact surface. The plurality of electrodes are disposed on the outer surface of the at least one flexible circuit substrate. The flexible circuit assembly is positioned relative to the structural member such that a first set of electrodes of the plurality of electrodes is aligned with the first surface of the structural member and a second set of electrodes of the plurality of electrodes is aligned with the second surface of the structural member. The at least one flexible circuit substrate is coupled to at least one of the structural member and the at least one flexible circuit substrate.

### BRIEF DESCRIPTION OF THE DRAWINGS

FIG. 1 is a schematic and block diagram view of a catheter system incorporating various embodiments of the present disclosure.

FIG. 2 is a simplified diagrammatic and schematic view of an exemplary visualization, navigation, and/or mapping system of the catheter system shown in FIG. 1.

FIG. 3 is a perspective view of an exemplary electrode assembly suitable for use in the system of FIG. 1, illustrated in the form of a basket electrode assembly.

FIG. 4 is a perspective view of another exemplary electrode assembly suitable for use in the system of FIG. 1, illustrated in the form of a planar electrode assembly.

FIG. 5 is an end view of an exemplary spline suitable for use in the electrode assemblies shown in FIGS. 3 and 4.

FIG. 6 is a top view an exemplary subassembly of a flexible circuit assembly suitable for use in forming the spline of FIG. 5.

FIG. 7 illustrates a step in an exemplary method of forming the spline of FIG. 5.

FIG. 8 illustrates an end view of another exemplary spline suitable for use in the electrode assemblies shown in FIGS. 3 and 4.

FIG. 9 illustrates an exemplary flexible circuit assembly suitable for use in forming the spline of FIG. 8.

FIG. 10 illustrates a step in an exemplary method of forming the spline of FIG. 8.

FIG. 11 illustrates another, subsequent step in the exemplary method of forming the spline of FIG. 8.

FIG. 12 illustrates an end view of another exemplary spline suitable for use in the electrode assemblies shown in FIGS. 3 and 4.

FIG. 13 illustrates a step in an exemplary method of forming the spline of FIG. 12.

FIG. 14 is a perspective view of an exemplary subassembly in a spiral configuration suitable for use in the system shown in FIG. 1.

FIG. 15 is a flow diagram of an exemplary method of forming a spline for an electrode assembly, such as the electrode assemblies shown in FIGS. 3 and 4.

Corresponding reference characters indicate corresponding parts throughout the several views of the drawings. It is understood that that Figures are not necessarily to scale.

### DETAILED DESCRIPTION OF THE DISCLOSURE

The present disclosure relates generally to medical devices that are used in the human body. The present disclosure provides medical devices including splines for electrode assemblies for catheter systems, for use in the human vasculature for medical procedures such as mapping and/or ablation procedures, as well as methods of forming the splines. Electrode assemblies of the present disclosure include at least one spline including a structural member and a flexible circuit assembly. The flexible circuit assembly includes at least one flexible circuit substrate and a plurality of electrodes disposed on an outer surface of the at least one flexible circuit substrate. The flexible circuit assembly is positioned relative to the structural member such that electrodes are aligned with both a first surface and a second surface of the structural member. At least some known electrode assemblies include splines that are formed by positioning a structural member within a tubing material, and subsequently disposing electrodes on an outer surface of the tubing material.

Unlike some known electrode assemblies, the disclosed embodiments enable splines to be formed by coupling electrodes directly to one or more surfaces of the structural member via the flexible circuit substrates, thereby removing the need for intermediate tubing material. Further, the disclosed embodiments enable electrodes to be disposed on two or more surfaces of a single spline, thereby enabling more electrodes to be disposed on a single spline. Such an arrangement improves electrode density about the circumference of the electrode assembly, which can improve the precision of mapping and/or ablation procedures and, therefore, lead to more consistent and improved patient outcomes.

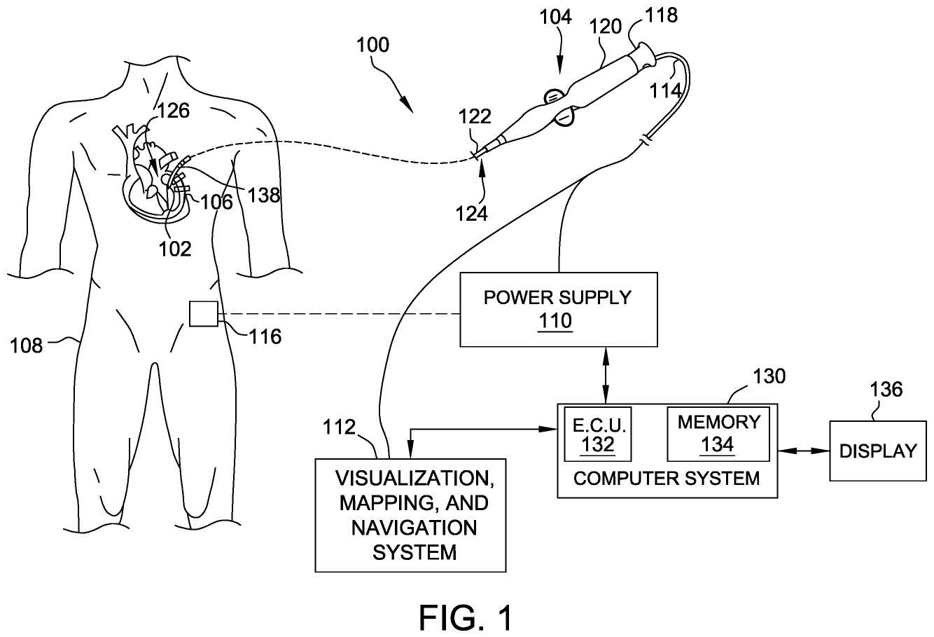

Referring now to the drawings, FIG. 1 is a schematic and block diagram view of a catheter system 100 suitable for diagnostic purposes, anatomical mapping and/or ablation therapy (e.g., electroporation therapy). In general, the various embodiments include an electrode assembly disposed at the distal end of a catheter shaft. As used herein, “proximal” refers to a direction toward the end of the catheter near the clinician and “distal” refers to a direction away from the clinician and (generally) inside the body of an individual. The electrode assembly includes one or more individual, electrically-isolated electrode elements. Each electrode element, also referred to herein as a catheter electrode, is individually wired such that it can be selectively paired or combined with any other electrode element to act as a bipolar or a multi-polar electrode.

System 100 may be used for irreversible electroporation to destroy tissue. In particular, system 100 may be used for electroporation-induced primary necrosis therapy, which refers to the effects of delivering electrical current in such manner as to directly cause an irreversible loss of plasma membrane (cell wall) integrity leading to its breakdown and cell necrosis. This mechanism of cell death may be viewed as an “outside-in” process, meaning that the disruption of the outside wall of the cell causes detrimental effects to the inside of the cell. Typically, for classical plasma membrane electroporation, electric current is delivered as a pulsed electric field (i.e., pulsed field ablation (PFA)) in the form of short-duration pulses (e.g., 0.1 to 20 ms duration) between closely spaced electrodes capable of delivering an electric field strength of about 0.1 to 1.0 kV/cm.

System 100 includes an electrode assembly 102 including at least one catheter electrode configured to be used as described below. Electrode assembly 102 is incorporated as part of a medical device such as a catheter 104 for electroporation therapy, diagnostic, mapping, and/or therapeutic procedures. For example, electrode assembly 102 may be used for mapping one or more structures 106 within a patient's body 108, also referred to herein as internal body structures 106. As another example, electrode assembly 102 may be used for ablation therapy (e.g., electroporation therapy) of tissue of structures 106 in body 108. In the illustrated embodiment, structure 106 includes a patient's vasculature and/or heart or cardiac tissue. It should be understood, however, that embodiments may be used to conduct mapping, diagnosis, and/or ablation therapy with respect to a variety of other body structures and/or tissues.

System 100 also includes additional sub-systems, such as a power supply 110 and a visualization, navigation, and mapping system 112 for visualization, mapping and navigation of internal body structures 106. Power supply 110 is any power supply configured to energize or excite electrodes of electrode assembly 102 and/or generate an electrical and/or magnetic field to perform a suitable function during a medical procedure. For example, power supply 110 includes radiofrequency (RF) ablation and/or electroporation generators to allow system 100 to be used for RF ablation and electroporation procedures. In such embodiments, power supply 110 is configured to energize the electrodes in accordance with an ablation strategy, which may be predetermined or may be user-selectable. When used for RF ablation procedures, power supply 110 outputs radio frequency (RF) energy to catheter 104 through a cable 114. The RF energy leaves catheter 104 through electrodes of electrode assembly 102 (e.g., using bi-polar electrode stimulation). The dissipation of the RF energy in the body increases the temperature near the electrodes, thereby permitting RF ablation to occur.

In some embodiments, system 100 includes one or more return electrodes 116 (e.g., patch electrodes) for mono-polar electrode stimulation or to perform mapping functions, as described further herein. In such embodiments, power supply 110 includes a signal generator coupled to patch electrodes 116 and configured to excite patch electrode 116 to generate an electrical field within body 108.

In the illustrated embodiment, catheter 104 includes a cable connector or interface 118, a handle 120, and a shaft 122 having a proximal end 124 and a distal end 126. Catheter 104 may also include other conventional components not illustrated herein such as one or more sensors (e.g., sensors 138), additional electrodes, and corresponding conductors or leads. Connector 118 provides mechanical and electrical connection(s) for cable 114 extending from power supply 110 and/or visualization, navigation, and mapping system 112, and as shown is disposed at the proximal end of catheter 104.

Handle 120 provides a location for the clinician to hold catheter 104 and may further provide means for steering or guiding shaft 122 within body 108. For example, handle 120 may include means to change the length of one or more guidewires extending through catheter 104 to distal end 126 of shaft 122 or other means to steer shaft 122. Moreover, in some embodiments, handle 120 may be configured to vary the shape, size, and/or orientation of a portion of the catheter. It will be understood that the construction of handle 120 may vary. In an alternative exemplary embodiment, catheter 104 may be robotically driven or controlled. Accordingly, rather than a clinician manipulating a handle to advance/retract and/or steer or guide catheter 104 (and shaft 122 thereof in particular), a robot is used to manipulate catheter 104.

Shaft 122 is an elongated, tubular, flexible member configured for movement within body 108. Shaft 122 is configured to support electrode assembly 102 as well as contain associated conductors, and possibly additional electronics used for signal processing or conditioning. Shaft 122 may also permit transport, delivery and/or removal of fluids (including irrigation fluids and bodily fluids), medicines, and/or surgical tools or instruments. Shaft 122 may be made from conventional materials such as polyurethane and defines one or more lumens configured to house and/or transport electrical conductors, fluids or surgical tools. Shaft 122 may be introduced into a blood vessel or other structure 106 within body 108 through a conventional introducer. Shaft 122 may then be advanced, retracted and/or steered or guided through body 108 to a desired location within structure 106, including through the use of guidewires or other means known in the art.

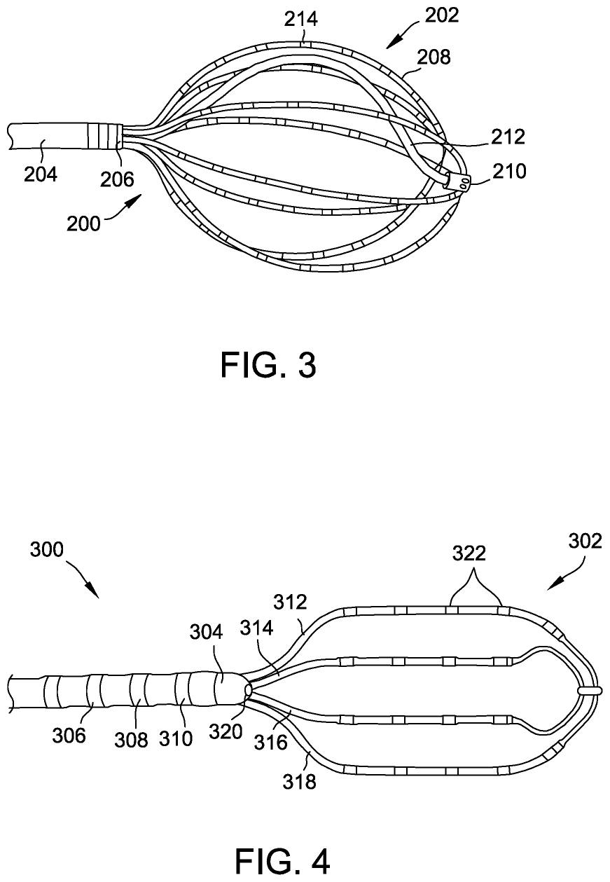

In embodiments of the present disclosure, electrode assembly 102 is coupled to distal end 126 of shaft 122 for delivery of electrode assembly 102 to a target location within the patient's body 108. In some embodiments, electrode assembly 102 is an electrode basket that is selectively configurable between a collapsed configuration and an expanded configuration. For example, electrode assembly 102 may be delivered to the target location in the collapsed configuration (e.g., within catheter shaft 122 and/or within a separate guide tube, not specifically shown). In this example, electrode assembly 102 is then deployed in the expanded configuration at the target location to perform a medical procedure (e.g., an ablation or mapping procedure). In some embodiments, electrode assembly 102 is in the form of a planar or grid electrode assembly that includes a paddle coupled to a catheter body. In embodiments of the present disclosure, electrode assembly 102 is subsequently energized using power supply 110 to perform the medical procedure at the target location. Electrode assembly 102 may include a plurality of electrodes (e.g., electrodes 226, shown in FIGS. 3-5) thereon. Electrode assembly 102 and/or catheter shaft 122 may include one or more sensors 138 therein or thereon.

Sensors 138 mounted in or on shaft 122 and/or in or on electrode assembly 102 may be provided for a variety of diagnostic and therapeutic purposes including, for example, electrophysiological studies and cardiac mapping. In an exemplary embodiment, one or more of the sensors 138 are provided to perform a position sensing function. More particularly, one or more of the sensors 138 are configured to be positioning sensors that provide information to, for example, the visualization, navigation, and mapping system 112 relating to the location (e.g., position and orientation) of the catheter 104, and the distal end 126 thereof, in particular, at certain points in time. Sensors 138 may comprise one of a number of types of sensors, such as, for example and without limitation, electrodes (e.g., tip electrodes and ring electrodes) or magnetic sensors (e.g., magnetic coils). It will be appreciated that the number, shape, orientation, and purpose of the sensors may vary.

Visualization, navigation, and mapping system 112 may be provided for visualization, mapping, and navigation of internal body structures 106, for example, by determining the position of electrode assembly 102, one or more splines, and/or specific electrodes thereon. These positions may be projected onto a geometrical anatomical model. Visualization, navigation, and mapping system 112 may include a conventional apparatus known generally in the art (e.g., an EnSite™ Velocity™ or EnSite Precision™ cardiac mapping and visualization system of Abbott Laboratories, or an EnSite™ NavX™ System, commercially available from Abbott Laboratories and as generally shown with reference to commonly assigned U.S. Pat. No. 7,263,397 titled “Method and Apparatus for Catheter Navigation and Location and Mapping in the Heart,” the entire disclosure of which is incorporated herein by reference). Other systems and components suitable for use with visualization, navigation, and mapping system 112 are described, for example, in U.S. Pat. No. 7,885,707 titled “Method of Scaling Navigation Signals to Account for Impedance Drift in Tissue” and U.S. Patent Application Publication No. 2018/0296111 titled “Orientation Independent Sensing, Mapping, Interface and Analysis System and Methods”, the entire disclosures of which are incorporated herein by reference. In various embodiments, visualization, navigation, and mapping system 112 uses the electrodes of electrode assembly 102 as bipolar pairs for visualization, mapping, and navigation of internal body structures 106. It should be understood, however, that this system is exemplary only and not limiting in nature. Other technologies for visualizing/navigating/mapping a catheter in space are known, including, for example, the CARTO navigation and location system of Biosense Webster, Inc., the AURORA® system of Northern Digital Inc., commonly available fluoroscopy systems, or a magnetic location system such as the gMPS system from MediGuide Ltd. In this regard, some of the localization, navigation and/or visualization system would provide a sensor for producing signals indicative of catheter location information, and may include, for example one or more electrodes in the case of an impedance-based localization system, or alternatively, one or more coils (i.e., wire windings) configured to detect one or more characteristics of a magnetic field, for example in the case of a magnetic-field based localization system.

System 100 may further include a main computer system 130, which may be integrated with visualization, navigation, and mapping system 112 in certain embodiments. Computer system 130 may include an electronic control unit (ECU) 132 and a memory 134. Computer system 130 further includes a display device 136, which may be integral to computer system 130 and/or coupled thereto. Catheter 104, and therefore electrode assembly 102, may be coupled to computer system 130 and/or visualization, navigation, and mapping system 112 with a wired or wireless connection.

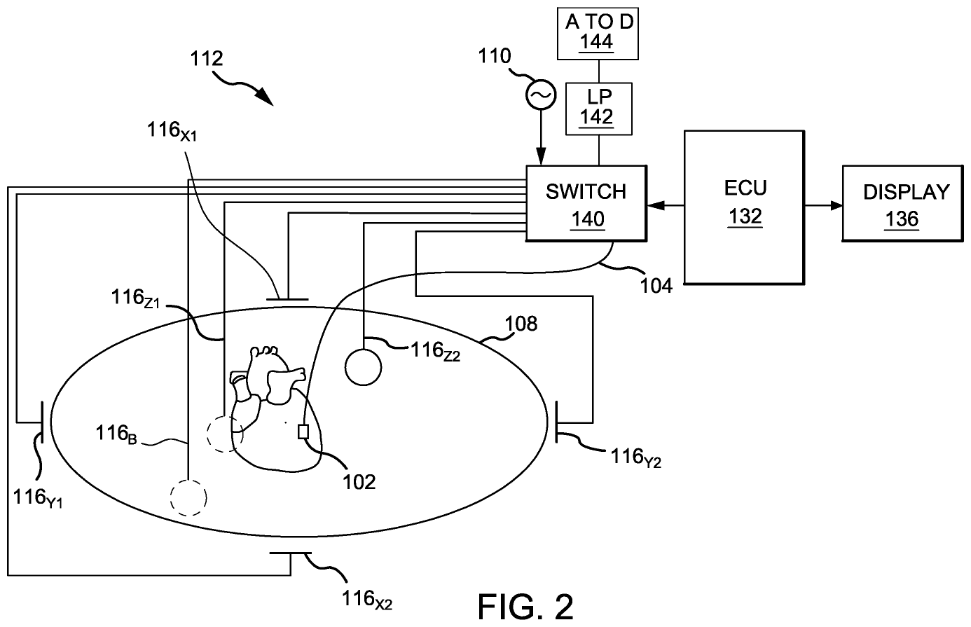

FIG. 2 is a simplified diagrammatic and schematic view of visualization, navigation, and/or mapping system 112 of system 100 (shown in FIG. 1). With reference to FIGS. 1 and 2, visualization, navigation, and mapping system 112 may include a plurality of patch electrodes 116, ECU 132, and display device 136, among other components. With the exception of a patch electrode 116_{B} called a “belly patch,” patch electrodes 116 are provided to generate electrical signals used, for example, in determining the position and orientation of catheter 104, and in the guidance thereof In one embodiment, patch electrodes 116 are placed orthogonally on the surface of the patient's body 108 and are used to create axes-specific electric fields within the body 108. For instance, in one exemplary embodiment, patch electrodes 116_{X1}, 116_{X2} may be placed along a first (x) axis. Patch electrodes 116_{Y1}, 116_{Y2} may be placed along a second (y) axis, and patch electrodes 116_{Z1}, 116_{Z2} may be placed along a third (z) axis. In other embodiments, the dipoles created may not be on an axis, for example, a dipole between electrodes 116_{X1} and 116_{Y1}. Each of the patch electrodes 116 may be coupled to a multiplex switch 140. In an exemplary embodiment, ECU 132 is configured through appropriate software to provide control signals to switch 140 to thereby sequentially couple pairs of electrodes 116 to a signal generator (e.g., power supply 110). Excitation of each pair of electrodes 116 generates an electrical field within body 108 and within an area of interest such as the patient's heart. Voltage levels at non-excited electrodes 116, which are referenced to the belly patch 116_{B}, are filtered by, for example, a low pass filter 142, converted by an analog-to-digital converter 144, and provided to ECU 132 for use as reference values.

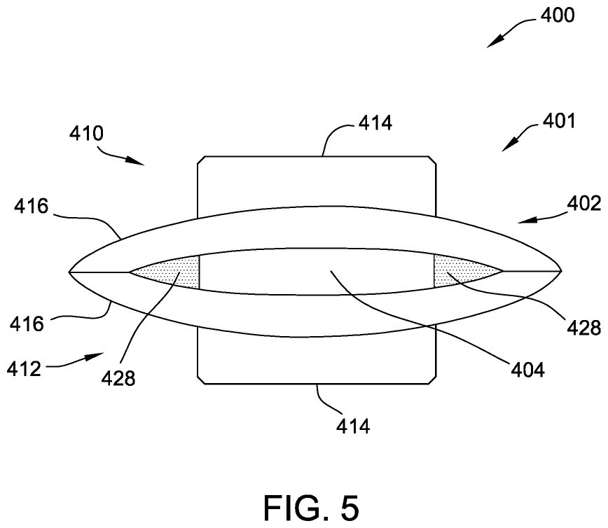

As discussed above, catheter 104 includes electrode assembly 102 coupled thereto. In the exemplary embodiment, electrode assembly 102 includes a plurality of splines, each including one or more electrodes (e.g., electrodes 414, shown in FIGS. 5-13) mounted therein or thereon that, in some embodiments, are electrically connected to power supply 110 and/or ECU 132 to provide one or more diagnostic or therapeutic purposes as described herein. In an exemplary embodiment, electrode assembly 102 is placed within electrical fields created in the body 108 by exciting patch electrodes 116. When disposed within the electric fields, the electrodes on electrode assembly 102 experience voltages that are dependent on their location between patch electrodes 116 and the position of each electrode relative to the tissue of the anatomic structure 106 being mapped. Voltage measurement comparisons made between each electrode on electrode assembly 102 and patch electrodes 116 can be used to determine the position of each electrode on electrode assembly 102 relative to the anatomic structure 106. This position information may then be used by ECU 132, for example, to generate models, such as surface models, and/or maps of, or corresponding to, the anatomic structure. Accordingly, as catheter 104 is moved along the surface of a desired anatomic structure 106, for example, electrode assembly 102 can be used to collect location data points that correspond to locations of the electrodes thereon, and therefore, the surface of the anatomic structure 106. These location data points can then be used by ECU 132, for example, to generate or construct a surface model of the anatomic structure. Further, information received from electrode assembly 102 can also be used to display on a display device, such as display device 136, the location and orientation of electrode assembly 102 and/or the tip of catheter 104. Accordingly, among other things, ECU 132 of visualization, navigation, and mapping system 112 provides a means for generating display signals used to control display device 136 and the creation of a graphical user interface (GUI) on display device 136.

ECU 132 may include, for example, a programmable microprocessor or microcontroller, or may comprise an application specific integrated circuit (ASIC). ECU 132 may include a central processing unit (CPU) and an input/output (I/O) interface through which ECU 132 may receive a plurality of input signals including, for example, signals generated by electrode assembly 102. ECU 132 may also generate a plurality of output signals including, for example, those used to control display device 136. ECU 132 may be configured to perform various functions, such as those described herein, with appropriate programming instructions or code. Accordingly, in one embodiment, ECU 132 is programmed with one or more computer programs encoded on a computer-readable storage medium for performing the functionality described herein.

ECU 132 may be configured to construct a geometrical anatomical model of structure 106 for display on display device 136. ECU 132 may also be configured to generate a GUI through which a user may, among other things, view a geometrical anatomical model and/or control electrode assembly 102. The anatomical model may include a 3-D model or a two-dimensional (2-D) model. To display the data and images that are produced by ECU 132, display device 136 may include one or more conventional computer monitors or other display devices well known in the art.

FIG. 3 is a perspective view of an exemplary electrode assembly 102 suitable for use in system 100, illustrated in the form of a basket electrode assembly 200. Basket electrode assembly 200 includes a basket 202 coupled to a catheter body 204 (e.g., shaft 122) by a suitable proximal connector 206. Basket 202 includes a plurality of splines 208 and a distal coupler 210 at which each of splines 208 terminates. In some embodiments, such as the illustrated embodiment, basket electrode assembly 200 may also include irrigation tubing 212 (e.g., to supply fluids to basket electrode assembly 200). In other embodiments, irrigation tubing 212 may be omitted. Each of the plurality of splines 208 includes at least one electrode 214. In the illustrated embodiment, each of the plurality of splines includes eight electrodes 214, although each spline 208 may include more than or less than eight electrodes 214.

Electrodes 214 may be used for a variety of diagnostic and therapeutic purposes including, for example and without limitation, cardiac mapping and/or ablation (e.g., RF ablation or irreversible electroporation (IRE) ablation). For example, and in some embodiments, the electrode assembly 200 may be configured as a bipolar electrode assembly for use in bipolar-based electroporation therapy. Specifically, electrodes 214 may be individually electrically coupled to an electroporation generator, such as power supply 110 (e.g., via suitable electrical wire or other suitable electrical conductors extending through catheter shaft 122) and may be configured to be selectively energized (e.g., by power supply 110 and/or computer system 130) with opposite polarities to generate a potential and corresponding electric field therebetween, for IRE therapy. That is, one of electrodes 214 may be configured to function as a cathode, and another one of electrodes 214 may be configured to function as an anode. Electrodes 214 may be any suitable electroporation electrodes. Electrodes 214 may have any other shape or configuration. It is realized that the shape, size, and/or configuration of electrodes 214 may impact various parameters of the applied electroporation therapy. For example, increasing the surface area of one or more electrodes 214 may reduce the applied voltage needed to cause the same level of tissue destruction. In various embodiments, any combination of electrodes 214 may be configured as electrode pairs, including, for example and without limitation, adjacent electrodes, non-adjacent electrodes, electrodes on adjacent splines, electrodes on non-adjacent splines, and any other combination of electrodes that enables system 100 to function as described herein. In some embodiments, power supply 110 is configured to energize the electrodes in accordance with an ablation strategy, as described above.

FIG. 4 is a perspective view of another exemplary electrode assembly 102 suitable for use in system 100, illustrated in the form of a planar electrode assembly 300. Planar electrode assembly 300 includes a paddle 302 coupled to a catheter body 304 (e.g., shaft 122). In the illustrated embodiment, catheter body 304 includes body electrodes 306, 308, and 310 coupled thereto. In the illustrated embodiment, paddle 302 includes a first spline 312, a second spline 314, a third spline 316, and a fourth spline 318 coupled to catheter body 304 by a proximal coupler and coupled to each other by a distal connector at a distal end of paddle 302. In one embodiment, first spline 312 and fourth spline 318 can be one continuous segment, and second spline 314 and third spline 316 can be another continuous segment. In other embodiments the various splines can be separate segments coupled to each other. First spline 312, second spline 314, third spline 316, and fourth spline 318 are generally aligned in the same (topological) plane. Although paddle 302 is illustrated as relatively flat or planar in FIG. 4, it should be understood that paddle 302 may bend, curl, buckle, twist, and/or otherwise deform. Accordingly, the plane defined by paddle 302 and splines 312, 314, 316, and 318 may correspondingly deform, such that the plane is a non-flat topological plane. In the illustrated embodiment, planar electrode assembly 300 also includes an irrigation port 320 at a distal end of a catheter body 304. Irrigation port 320 is positioned to deliver an irrigant to a portion of one or more splines 312-318.

The plurality of splines can further comprise a varying number of electrodes 322. The electrodes in the illustrated embodiment can comprise single sided electrodes or electrodes printed on a flexible, bendable material. The electrodes may be evenly spaced along one or more surfaces of the splines. In other embodiments the electrodes can be evenly or unevenly spaced and the electrodes can comprise any other suitable type of electrodes.

Electrodes 322 may be used for a variety of diagnostic and therapeutic purposes including, for example and without limitation, cardiac mapping and/or ablation (e.g., RF ablation or IRE ablation). For example, and in some embodiments, the electrode assembly 300 may be configured as a bipolar electrode assembly for use in bipolar-based electroporation therapy. Specifically, electrodes 322 may be individually electrically coupled to an electroporation generator, such as power supply 110 (e.g., via suitable electrical wire or other suitable electrical conductors extending through catheter shaft 122) and may be configured to be selectively energized (e.g., by power supply 110 and/or computer system 130) with opposite polarities to generate a potential and corresponding electric field therebetween, for IRE therapy. That is, one of electrodes 322 may be configured to function as a cathode, and another one of electrodes 322 may be configured to function as an anode. Electrodes 322 may be any suitable electroporation electrodes. Electrodes 322 may have any other shape or configuration. It is realized that the shape, size, and/or configuration of electrodes 322 may impact various parameters of the applied electroporation therapy.

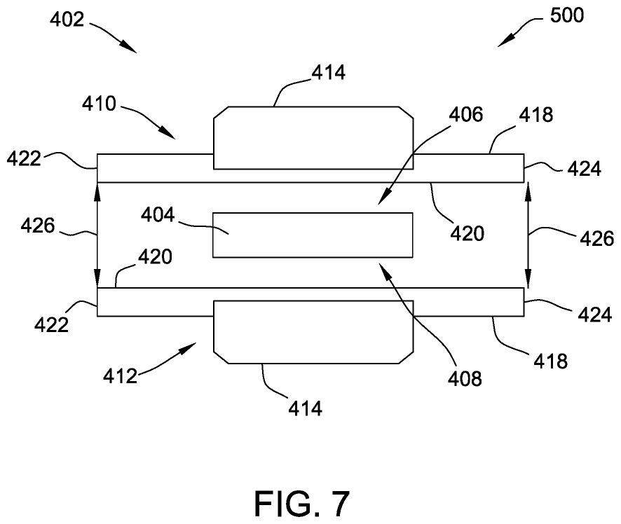

FIG. 5 illustrates an end view of an exemplary spline 400 suitable for use in the electrode assemblies described herein (e.g., electrode assemblies 102, 200, and 300). In particular, spline 400 may be incorporated into basket electrode assembly 200 as one or more of splines 208 (both shown in FIG. 3). Additionally or alternatively, spline 400 may be incorporated into planar electrode assembly 300 as one or more of splines 312, 314, 316, and 318 (all shown in FIG. 4). In the illustrated embodiment, spline 400 includes a flexible circuit assembly 402 and a structural member 404. Structural member 404 extends from a proximal end 401 to a distal end (not shown in FIG. 5) of spline 400, and generally provides structural support to spline 400 and components thereof (e.g., flexible circuit assembly 402). Structural member 404 includes a first surface 406 and a second surface 408 (both shown in FIG. 7). In the illustrated embodiment, structural member 404 has a rectangular cross-section, and first surface 406 and second surface 408 are located on opposing sides of structural member 404. In other embodiments, structural member 404 may have a cross-sectional shape other than rectangular, and first surface 406 and second surface 408 may be on non-opposing sides of structural member 404. Further, in the exemplary embodiment, structural member 404 is a single, continuous member that extends the entire length of spline 400 (i.e., from proximal end 401 to distal end).

Structural member 404 may be constructed from a variety of suitable materials including, for example and without limitation, metal alloys, stainless steel, copper-aluminum-nickel alloys, alloys including zinc, copper, gold, and/or iron, polymers including any of the above materials, shape memory polymers, and/or combinations thereof. In some embodiments, structural member 404 may be constructed from a non-metallic material, such as, for example, a formed stiff plastic material. In an exemplary embodiment, structural member 404 is constructed of a shape memory alloy. One particularly preferred shape memory alloy for use is Nitinol, a nickel-titanium (NiTi) alloy. Nitinol is an approximately stoichiometric alloy of nickel and titanium, which may also include minor amounts of other metals to achieve desired properties. Nickel-titanium alloys are very elastic and are commonly referred to as “superelastic” or “pseudoelastic.” Such memory-shape alloys tend to have a temperature induced phase change that will cause the material to have a preferred configuration that can be fixed by heating the material above a certain transition temperature to induce a change in the phase of the material. When the alloy is cooled back down, the alloy will “recall” the shape it was in during the heat treatment and will tend to assume that configuration unless constrained from doing so.

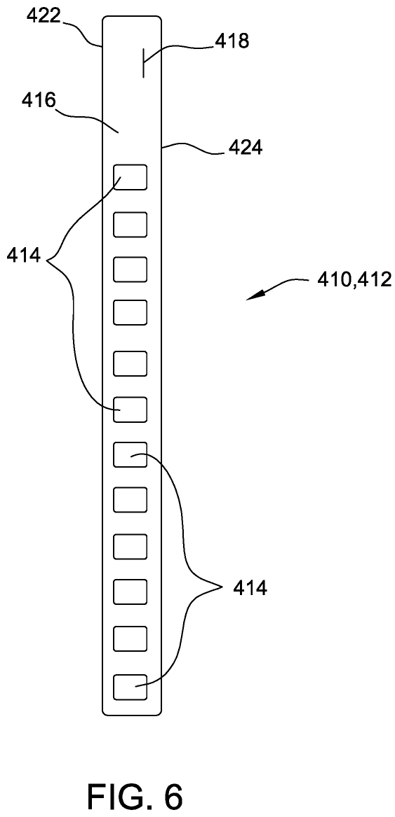

Flexible circuit assembly 402 includes at least one flexible circuit designed to be bent or flexed in use, and is typically implemented on a flexible substrate. In the illustrated embodiment, flexible circuit assembly 402 includes a first subassembly 410 and a second subassembly 412. FIG. 6 is a top view of each of the first and second subassemblies of flexible circuit assembly 402 (shown in FIG. 5). In the illustrated embodiment, first subassembly 410 and second subassembly 412 are identical, although in other embodiments, first subassembly 410 and second subassembly 412 may have different configurations. With reference to FIGS. 5 and 6, each of first subassembly 410 and second subassembly 412 includes a plurality of electrodes 414 disposed on a flexible circuit substrate 416. Each flexible circuit substrate 416 of subassemblies 410, 412 includes an outer surface 418 opposite a contact surface (e.g., inner surface) 420 (shown in FIG. 7). Each flexible circuit substrate 416 also includes a first longitudinal edge 422 and a second longitudinal edge 424. In one embodiment, flexible circuit substrate 416 is a flexible printed circuit, such as a polyimide flexible circuit. In one example, flexible circuit substrate 416 may be a Kapton® polyimide flexible circuit.

Electrodes 414 are disposed on outer surface 418 of each flexible circuit substrate 416. Electrodes 414 may be any suitable type of electrodes, such as single sided electrodes disposed on outer surface 418 or electrodes printed on flexible circuit substrate 416. In an exemplary embodiment, first and second subassemblies 410, 412 each include a single flexible circuit that includes electrodes on one side of the flexible circuit. The illustrated embodiment includes 12 electrodes disposed on outer surface 418, although other embodiments may include more than or fewer than 12 electrodes. For example, first subassembly 410 and second subassembly 412 may include any suitable number of electrodes that enables system 100 to function as described herein. In one example, spline 400 may include eight to twelve electrodes on each of subassemblies 410, 412. Additionally, the electrodes 414 are rectangular or pseudo-rectangular (i.e., rounded rectangles) in the illustrated embodiment. In other embodiments, electrodes 414 may have any suitable shape or configuration that enables spline 400 to function as described herein, including, for example and without limitation, rounded or spherical. Plurality of electrodes 414 of first subassembly 410 may be interchangeably referred to herein as a first set of electrodes, and plurality of electrodes 414 of second subassembly 412 may be interchangeably referred to herein as a second set of electrodes.

FIG. 7 illustrates a step 500 in an exemplary method of forming spline 400 (shown in FIG. 5). With reference to FIGS. 5-7, the exemplary method of forming spline 400 includes positioning structural member 404 between first subassembly 410 and second subassembly 412 such that electrodes 414 of first subassembly 410 (e.g., the first set of electrodes) are aligned with first surface 406 of structural member 404 and electrodes 414 of second subassembly 412 (e.g., the second set of electrodes) are aligned with second surface 408 of structural member 404. The exemplary method further includes coupling flexible circuit substrates 416 to one another at the respective first edges 422 and at the respective second edges 424, as illustrated by arrows 426 in FIG. 7. Flexible circuit substrates 416 may be coupled at the respective first edges 422 and at the respective second edges 424 using an adhesive material 428 (shown in FIG. 5). In one embodiment, openings (e.g., gaps of space) formed by coupling the respective first edges 422 and the respective second edges 424 may be entirely filled with adhesive material 428. Adhesive material 428 may be applied on contact surface 420 of one or both flexible circuit substrates 416. Adhesive material 428 may be a biocompatible adhesive or any suitable material for adhering flexible circuit substrates 416 together. In other embodiments, flexible circuit substrates 416 are heat sealed together.

In some embodiments, flexible circuit substrates 416 are coupled only to one another, and are not fixed to structural member 404. For example, flexible circuit substrates 416 of first and second subassemblies 410 and 412 may be coupled only at the respective first and second edges 422, 424 such that the coupled flexible circuit substrates 416 are free to move and slide relative to structural member 404. In other embodiments, one or both of flexible circuit substrates 416 are coupled directly to structural member 404 at first surface 406 and/or second surface 408. In the exemplary embodiment, structural member 404 is sandwiched between two separate subassemblies 410, 412 to form a double sided spline of electrodes 414.

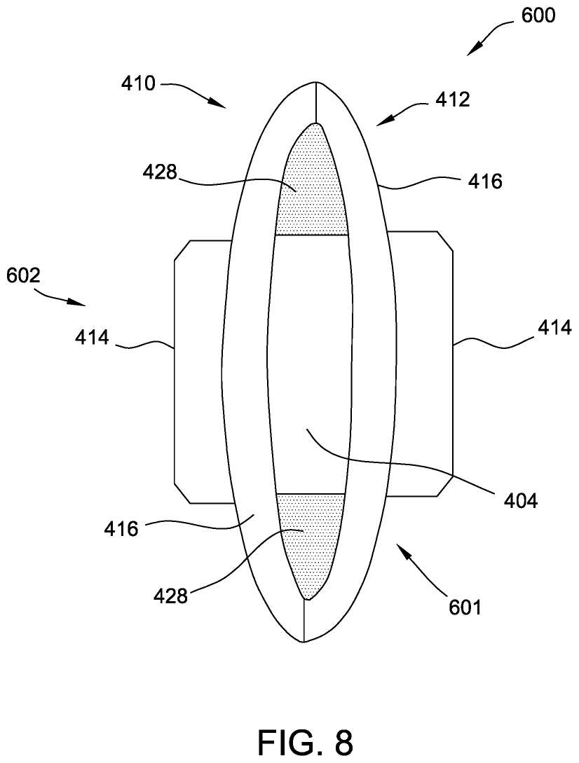

FIG. 8 illustrates an end view of another exemplary spline 600 suitable for use in electrode assembly 102 (shown in FIG. 1). In particular, spline 600 may be incorporated into basket electrode assembly 200 as one or more of splines 208 (both shown in FIG. 3). Additionally or alternatively, spline 600 may be incorporated into planar electrode assembly 300 as one or more of splines 312, 314, 316, and 318 (all shown in FIG. 4). Spline 600 may be substantially similar to or have substantially the configuration as spline 400, as described above. Spline 600 includes structural member 404 and a flexible circuit assembly 602. Structural member 404 extends from a proximal end 601 to a distal end (not shown) of spline 600. Flexible circuit substrates 416 may be coupled together using an adhesive material 428. In one embodiment, openings (e.g., gaps of space) formed by coupling flexible circuit substrates 416 may be entirely filled with adhesive material 428.

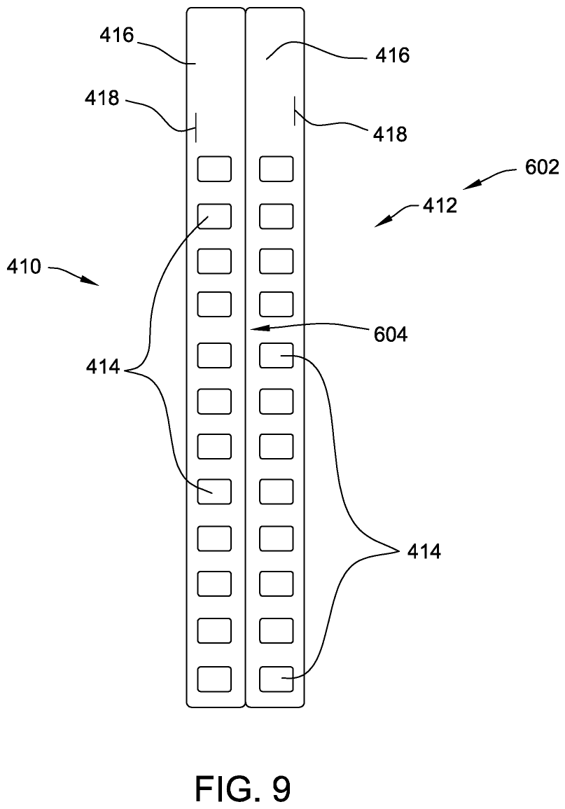

FIG. 9 is a top view of flexible circuit assembly 602 of spline 600 (shown in FIG. 8). With reference to FIGS. 8 and 9, flexible circuit assembly 602 includes first and second subassemblies 410, 412. Each of subassemblies 410, 412 includes a plurality of electrodes 414 disposed on outer surface 418 of flexible circuit substrate 416. In the embodiment illustrated in FIGS. 8 and 9, first subassembly 410 and second subassembly 412 of flexible circuit assembly 602 are joined together at a fold line 604. Fold line 604 may include any line of weakness that facilitates folding or bending of subassemblies 410, 412 joined at fold line 604, such as, for example, a score line, a break line, a crease, a perforated line, and combinations thereof.

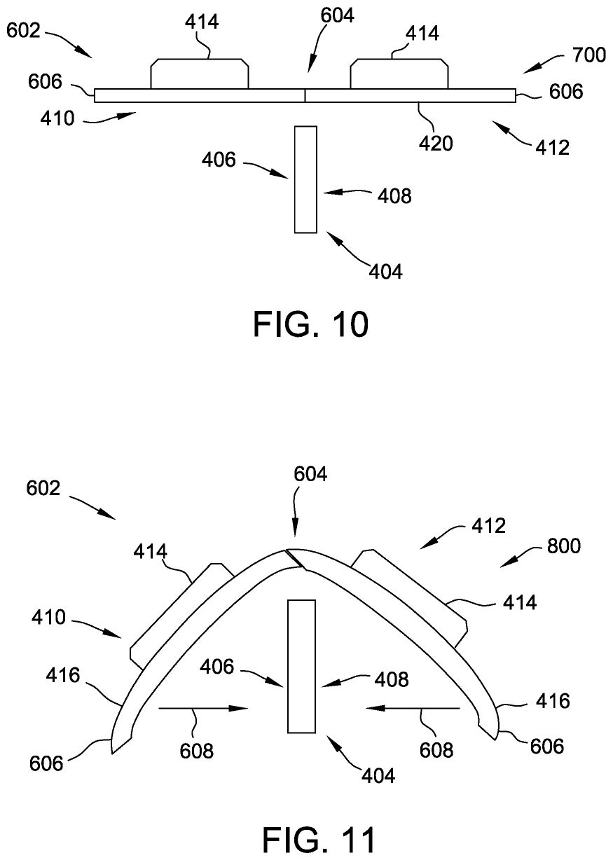

FIG. 10 illustrates a step 700 in an exemplary method of forming spline 600 (shown in FIG. 8) using flexible circuit assembly 602. As shown in FIG. 10, the exemplary method of forming spline 600 includes positioning structural member 404 adjacent subassemblies 410, 412 near contact surface 420 such that structural member 404 is proximate fold line 604. FIG. 11 illustrates another, subsequent step 800 in the exemplary method of forming spline 600. As shown in FIG. 11, the exemplary method of forming spline 600 also includes folding flexible circuit assembly 602 about fold line 604 and around structural member 404 such that electrodes 414 of first subassembly 410 (e.g., the first set of electrodes) are coupled proximate to first surface 406 of structural member 404 and electrodes 414 of second subassembly 412 (e.g., the second set of electrodes) are coupled proximate to second surface 408 of structural member 404. The exemplary method of forming spline 600 also includes coupling flexible circuit substrates 416 of subassemblies 410, 412 to one another at longitudinal edges 606 of flexible circuit assembly 602, as illustrated by arrows 608 in FIG. 10. As shown in FIG. 8, flexible circuit substrates 416 may be coupled at edges 606 using adhesive material 428. In some embodiments, flexible circuit substrates 416 of first and second subassemblies 410, 412 are coupled only to one another, and are not fixed to structural member 404. For example, flexible circuit substrates 416 of first and second subassemblies 410 and 412 may be coupled only at longitudinal edges 606 such that the coupled flexible circuit substrates 416 are free to move and slide relative to structural member 404. In other embodiments, one or both of flexible circuit substrates 416 are coupled directly to structural member 404 at first surface 406 and/or second surface 408.

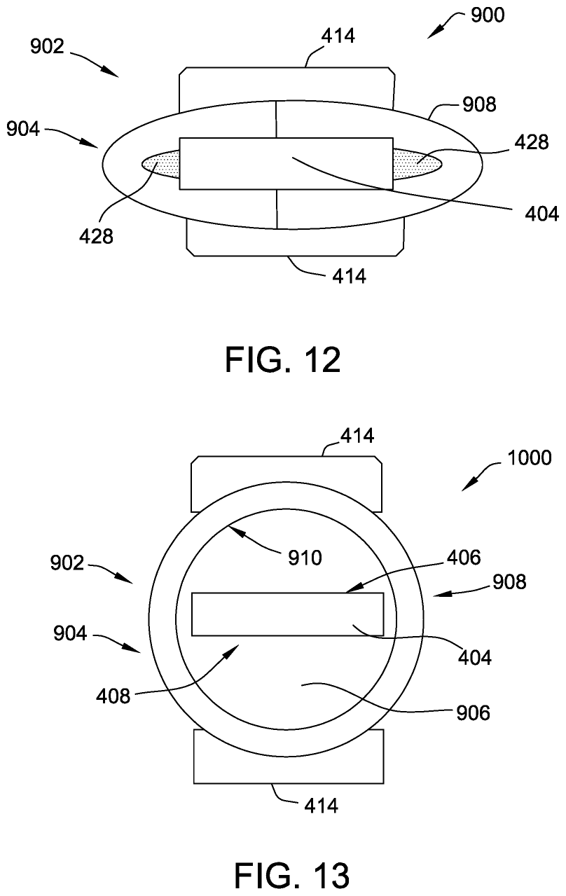

FIG. 12 illustrates an end view of another exemplary spline 900 suitable for use in electrode assembly 102 (shown in FIG. 1). In particular, spline 900 may be incorporated into basket electrode assembly 200 as one or more of splines 208 (both shown in FIG. 3). Additionally or alternatively, spline 900 may be incorporated into planar electrode assembly 300 as one or more of splines 312, 314, 316, and 318 (all shown in FIG. 4). Spline 900 includes structural member 404 and a flexible circuit assembly 902. Structural member 404 is described above with respect to spline 400 (shown in FIG. 5) and spline 600 (shown in FIG. 8). FIG. 13 illustrates a step 1000 in an exemplary method of forming spline 900 (shown in FIG. 12). With reference to FIGS. 12 and 13, flexible circuit assembly 902 of spline 900 includes a flexible tubular substrate 904 that defines a cavity 906 therein. In one embodiment, flexible tubular substrate 904 is a flexible printed circuit formed in the shape of a compressible, cylindrical tube.

A plurality of electrodes 414 is disposed on an outer surface 908 of flexible tubular substrate 904. Electrodes 414 may have the same configuration as described above with reference to spline 400 (shown in FIG. 5) and spline 600 (shown in FIG. 8). Flexible circuit assembly 902 includes two sets (a first set and a second set) of electrodes 414 disposed on opposite sides of flexible tubular substrate 904. Each set of electrodes 414 may include any suitable number of electrodes 414 that enables spline 900 to function as described herein. For example, each set of electrodes 414 may include between eight and twelve electrodes.

As shown in FIG. 13, the exemplary method of forming spline 900 includes inserting structural member 404 in cavity 906 of flexible tubular substrate 904. The exemplary method of forming spline 900 further includes compressing flexible tubular substrate 904 (e.g., by applying force to outer surface 908) such that the first set of electrodes 414 of flexible circuit assembly 902 is coupled proximate to first surface 406 of structural member 404, and the second set of electrodes 414 of flexible circuit assembly 902 is coupled proximate to second surface 408 of structural member 404. Adhesive material 428 may be applied on a contact surface (e.g., an inner surface) 910 of flexible tubular substrate 904 such that, when flexible tubular substrate 904 is compressed, contact surface 910 of flexible tubular substrate 904 adheres to structural member 404. In one embodiment, openings (e.g., gaps of space) formed by compressing flexible tubular substrate 904 may be entirely filled with adhesive material 428.

Although the splines and spline-forming methods of the present disclosure are described with reference to specific electrode assemblies (e.g., basket electrode assembly 200 and planar electrode assembly 300), it should be understood that the disclosed splines and spline-forming methods are not limited to use in the specific electrode assembly constructions shown and described herein, and may be incorporated in any other suitable electrode assembly that enables system 100 (shown in FIG. 1) to function as described herein.

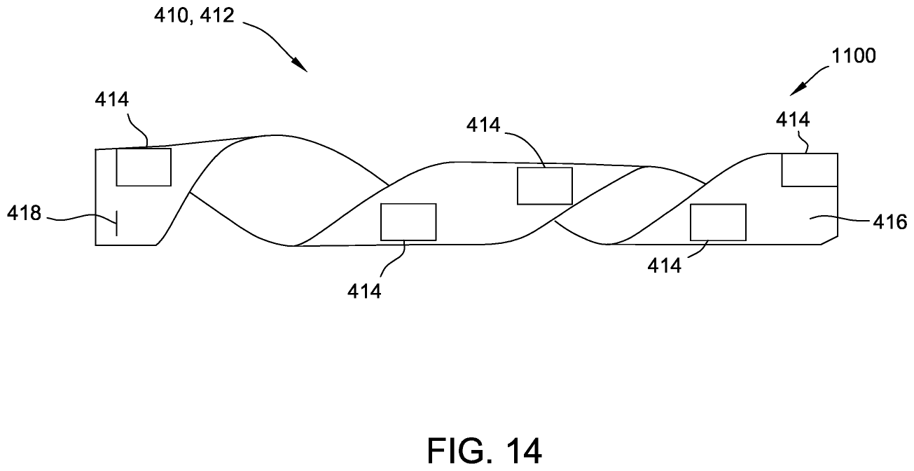

FIG. 14 is a perspective view of one of subassemblies 410, 412 arranged in a spiral configuration 1100 suitable for use in system 100 (shown in FIG. 1). In the illustrated embodiment, structural member 404 is omitted. In other embodiments, the individual subassembly 410, 412 may be coupled to a structural member, such as structural member 404, to facilitate deploying the individual subassembly 410, 412 into a desired spiral shape or other desired shape, for example, to facilitate contact with certain anatomical structures. Individual subassembly 410, 412 is rolled along a length of subassembly 410, 412 to form spiral configuration 1100, and lengthened to reduce an outer diameter of spiral configuration 1100. More specifically, in these embodiments, flexible circuit substrate 416 of one subassembly 410, 412 may be rolled into a coil and lengthened for insertion as one long linear catheter in system 100. In the illustrated embodiment, electrodes 414 are disposed on outer surface 418 of flexible circuit substrate 416. Electrodes 414 may be any suitable type of electrodes, such as single sided electrodes disposed on outer surface 418 or electrodes printed on flexible circuit substrate 416. In some embodiments, individual subassembly 410, 412 including structural member 404 may be rolled into a spiral configuration.

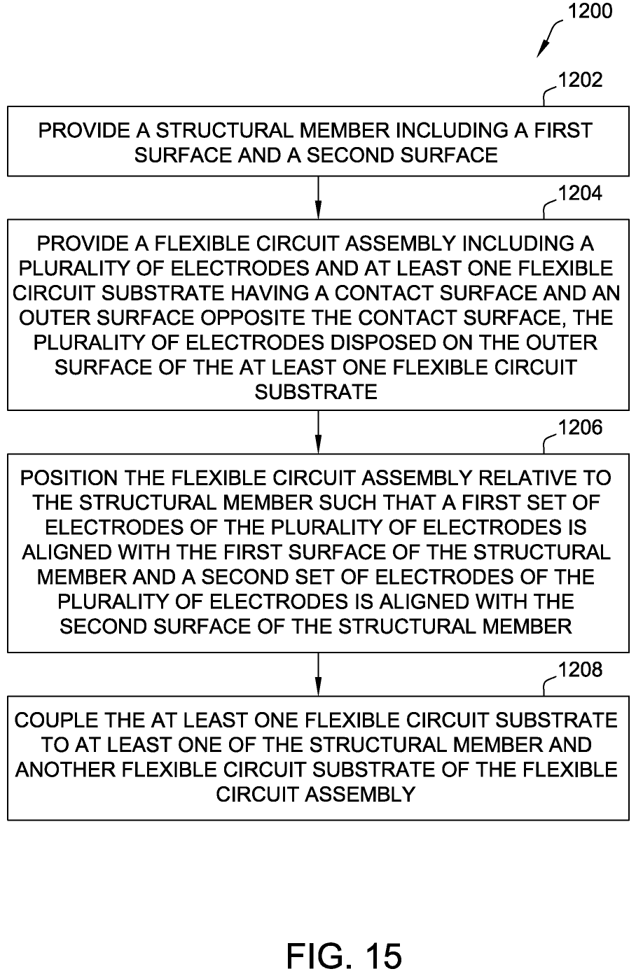

FIG. 15 is a flow diagram of an exemplary method 1200 of forming a spline, such as spline 400 (shown in FIG. 5), spline 600 (shown in FIG. 8), or spline 900 (shown in FIG. 12) for use in an electrode assembly (e.g., electrode assembly 102, shown in FIG. 1). Method 1200 includes providing 1202 a structural member (e.g., structural member 404) that includes a first surface and a second surface. Method 1200 also includes providing 1204 a flexible circuit assembly (e.g., flexible circuit assembly 402, flexible circuit assembly 602, or flexible circuit assembly 902) that includes a plurality of electrodes and at least one flexible circuit substrate (e.g., flexible circuit substrate 416 or flexible tubular substrate 904). The at least one flexible circuit substrate includes a contact surface and an outer surface opposite the contact surface. The plurality of electrodes is disposed on the outer surface of the at least one flexible circuit substrate. Method 1200 also includes positioning 1206 the flexible circuit assembly relative to the structural member such that a first set of electrodes of the plurality of electrodes is aligned with the first surface of the structural member and a second set of electrodes of the plurality of electrodes is aligned with the second surface of structural member. Method 1200 also includes coupling 1208 the at least one flexible circuit substrate to at least one of the structural member and the at least one flexible circuit substrate.

Although certain steps of the example method are numbered, such numbering does not indicate that the steps must be performed in the order listed. Thus, particular steps need not be performed in the exact order they are presented, unless the description thereof specifically require such order. The steps may be performed in the order listed, or in another suitable order.

Although the embodiments and examples disclosed herein have been described with reference to particular embodiments, it is to be understood that these embodiments and examples are merely illustrative of the principles and applications of the present disclosure. It is therefore to be understood that numerous modifications can be made to the illustrative embodiments and examples and that other arrangements can be devised without departing from the spirit and scope of the present disclosure as defined by the claims. Thus, it is intended that the present application cover the modifications and variations of these embodiments and their equivalents.

This written description uses examples to disclose the invention, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A method of forming a spline for an electrode assembly for a catheter system, the method comprising:
providing a structural member including a first surface and a second surface;
providing a flexible circuit assembly including a plurality of electrodes and at least one flexible circuit substrate having a contact surface and an outer surface opposite the contact surface, the plurality of electrodes disposed on the outer surface of the at least one flexible circuit substrate;
positioning the flexible circuit assembly relative to the structural member such that a first set of electrodes of the plurality of electrodes is aligned with the first surface of the structural member and a second set of electrodes of the plurality of electrodes is aligned with the second surface of the structural member; and
coupling the at least one flexible circuit substrate to at least one of the structural member and the at least one flexible circuit substrate of the flexible circuit assembly.

2. The method of claim 1, wherein coupling the at least one flexible circuit substrate to at least one of the structural member and the at least one flexible circuit substrate comprises coupling the at least one flexible circuit substrate to the structural member using an adhesive.

3. The method of claim 2, wherein coupling the at least one flexible circuit substrate to the structural member comprises coupling the contact surface of the at least one flexible circuit substrate to at least one of the first surface and the second surface of the structural member using the adhesive.

4. The method of claim 1, wherein coupling the at least one flexible circuit substrate to at least one of the structural member and the at least one flexible circuit substrate comprises heat sealing the at least one flexible circuit substrate to at least one of the structural member and the at least one flexible circuit substrate.

5. The method of claim 1, wherein the at least one flexible circuit substrate includes a first flexible circuit substrate and a second, separate flexible circuit substrate, each of the first and second flexible circuit substrates including first and second longitudinal edges;
wherein positioning the flexible circuit assembly relative to the structural member comprises positioning the structural member between the first and second flexible circuit substrates; and
wherein coupling the at least one flexible circuit substrate to at least one of the structural member and the at least one flexible circuit substrate comprises coupling the first and second flexible circuit substrates at their respective first longitudinal edges and at their respective second longitudinal edges such that the first set of electrodes of the plurality of electrodes is aligned with the first surface of the structural member and the second set of electrodes of the plurality of electrodes is aligned with the second surface of the structural member.

6. The method of claim 5, wherein coupling the first and second flexible circuit substrates at their respective first edges and at their respective second edges comprises coupling the first and second flexible circuit substrates using an adhesive such that the first and second flexible circuit substrates are slidable relative to the structural member.

7. The method of claim 1, wherein the flexible circuit assembly includes a first flexible circuit substrate and a second flexible circuit substrate joined to the first flexible circuit at a fold line, wherein forming the spline comprises folding the first flexible circuit substrate relative to the second flexible circuit substrate about the fold line and around the structural member such that the first set of electrodes is aligned with the first surface of the structural member and the second set of electrodes is aligned with the second surface of the structural member.

8. The method of claim 7, wherein coupling the at least one flexible circuit substrate to at least one of the structural member and the at least one flexible circuit substrate comprises coupling the first flexible circuit substrate to the second flexible circuit substrate using an adhesive.

9. The method of claim 1, wherein the at least one flexible circuit substrate of the flexible circuit assembly comprises a tubular substrate defining a cavity therein; and wherein positioning the flexible circuit assembly relative to the structural member comprises inserting the structural member in the cavity of the tubular substrate.

10. The method of claim 9, wherein positioning the flexible circuit assembly relative to the structural member further comprises compressing the tubular substrate such that the first set of electrodes is aligned with the first surface of the structural member and the second set of electrodes is aligned with the second surface of the structural member.

11. The method of claim 1, wherein the structural member is constructed of nitinol.

12. The method of claim 1, wherein the at least one flexible circuit substrate is a flexible printed circuit.

13. The method of claim 1, wherein the structural member includes a plurality of discrete members.

14. The method of claim 1, further comprising incorporating the formed spline into a planar electrode assembly.

15. The method of claim 1, further comprising incorporating the formed spline into a basket electrode assembly.

16. The method of claim 1, further comprising rolling the at least one flexible circuit substrate about a length of the at least one flexible circuit substrate to form a spiral configuration.

17. An electrode assembly for a catheter system, the electrode assembly having a longitudinal axis, a proximal end, and a distal end, the electrode assembly comprising:
at least one spline extending from the proximal end to the distal end of the electrode assembly, the at least one spline comprising:
a structural member extending from the proximal end to the distal end of the electrode assembly, the structural member including a first surface and a second surface; and
a flexible circuit assembly including a plurality of electrodes and at least one flexible circuit substrate having a contact surface and an outer surface opposite the contact surface, the plurality of electrodes disposed on the outer surface of the at least one flexible circuit substrate,
wherein the flexible circuit assembly is positioned relative to the structural member such that a first set of electrodes of the plurality of electrodes is aligned with the first surface of the structural member and a second set of electrodes of the plurality of electrodes is aligned with the second surface of the structural member, and wherein the at least one flexible circuit substrate is coupled to at least one of the structural member and the at least one flexible circuit substrate.

18. The electrode assembly of claim 17, wherein the flexible circuit assembly includes a first flexible circuit substrate and a second, separate flexible circuit substrate, each of the first and second flexible circuit substrates including first and second longitudinal edges;
wherein the structural member is positioned between the first and second flexible circuit substrates; and
wherein the first and second flexible circuit substrates are coupled at their respective first longitudinal edges and at their respective second longitudinal edges such that the first set of electrodes of the plurality of electrodes is aligned with the first surface of the structural member and the second set of electrodes of the plurality of electrodes is aligned with the second surface of the structural member.

19. The electrode assembly of claim 17, wherein the flexible circuit assembly includes at least two flexible circuit substrates joined at a fold line, wherein the flexible circuit assembly is folded about the fold line and around the structural member such that the first set of electrodes is aligned with the first surface of the structural member and the second set of electrodes is aligned with the second surface of the structural member.

20. A catheter system comprising:
a flexible catheter shaft;
a handle coupled to a proximal end of the catheter shaft;
an electrode assembly coupled to a distal end of the flexible catheter shaft and having a longitudinal axis, a proximal end, and a distal end, the electrode assembly comprising at least one spline extending from the proximal end to the distal end of the electrode assembly, the at least one spline comprising:
a structural member extending from the proximal end to the distal end of the electrode assembly, the structural member including a first surface and a second surface; and
a flexible circuit assembly including a plurality of electrodes and at least one flexible circuit substrate having a contact surface and an outer surface opposite the contact surface, the plurality of electrodes disposed on the outer surface of the at least one flexible circuit substrate, wherein the flexible circuit assembly is positioned relative to the structural member such that a first set of electrodes of the plurality of electrodes is aligned with the first surface of the structural member and a second set of electrodes of the plurality of electrodes is aligned with the second surface of the structural member, and wherein the at least one flexible circuit substrate is coupled to at least one of the structural member and the at least one flexible circuit substrate.
